(19) United States
(12) Patent Application Publication
(10) Pub. No.: US 2026/0033851 A1
(43) Pub. Date: Feb. 5, 2026

(54) WiPe n' Twirl

(71) Applicant: PATRICE IRIS ETHEL BARNER, ATLANTA, GA (US)

(72) Inventor: PATRICE IRIS ETHEL BARNER, ATLANTA, GA (US)

(21) Appl. No.: 18/793,906

(22) Filed: Aug. 4, 2024

(51) Int. Cl. A61B 17/24; A61B 17/00

(52) U.S. Cl. CPC A61B 17/24; A61B 2017/00398; A61B 2017/246; A61B 2217/007

(57) ABSTRACT

A hand-held electronic nose cleaning device with built-in wire lubrication and disposable cotton cleansers. The fluid filled tube possesses hypoallergenic fluid that provides effective cleansing for the nostrils. When the product is at rest, aside from nasal cleansing, this detachable hypoallergenic fluid tube provides hygienic care for various parts of the body.

### BACKGROUND OF THE INVENTION

### 1. Field of the Invention

This invention pertains to electronic nose cleaners.

More specifically, the invention the first hand-held, electronic nose cleaning device. The Wipe n′ Twirl includes a built-in lift-wire that supports the lift of a flexible-rubber lubrication tube powered by a CR2025 lithium battery, with respect to disposable cotton squares that receives the lubrication. The Wipe n′ Twirl rotates upward to the right into the nostrils to begin the process of deep cleansing and reverses the rotation to clear excess particles.

The invention possesses benefits and advantages that is revolutionary to the nasal care industry. In particular, the invention not only provides deep cleaning for the nostrils with accurately fitting lubricated cotton squares, but due to the 100 percent natural hypoallergenic fluid this invention contains, it cures allergies and sinus infections. The invention's flexible fluid filled tube is removable to complete the varied functions of the hypoallergenic fluid this invention contains. The invention hypoallergenic fluid heals cuts, heals burns, heals wounds, heals cold sores, rebuilds the health of hair, rebuilds healthy skin cells, removes make-up, and heals acne.

The invention is chic and trendy. This enables public use of the device, eliminating centuries of unsightly and discomforting methods for nose cleaning. For instance, the use of tissues, wet wipes, Q-Tip, and fingers are not suitable nor effective for public nor private deep cleansing of the nose. The invention replaces centuries of unsightly and discomforting nose cleaning methods with accurately fitting, self-cleansing, disposable cleansers.

### SUMMARY OF THE INVENTION

The WiPe n′ Twirl is a patent pending, revolutionary; soft grip, hand-held, electronic nose cleaning device, with built in wire lubrication and disposable cotton cleansers. The cleaning process begins with the light lubrication of the cotton square, exerted from a connected flexible tube filled with 100 percent natural hypoallergenic fluid upward-right rotation, that is rotated and lifted toward the nostrils by the lift wire. Excess particles are removed from the nostrils with the reversed left rotation of the tube and connected square cotton.

The fluid filled tube is the first item that is to be placed onto the mount of the lift-wire. Both members carry magnets at their base for easy attachment, detachment. The release of the fluid is initiated by a push of a right button which causes the tube to be constricted, thus releasing a small amount of fluid into the square cotton. The lift wire is also a rotating wire, its rotation is balanced on a track rotatory for accurate spot cleaning within the nostrils.

The cotton square is the second and last item to attach. This item is be attached to the fluid filled tube. The cotton square has a dual sided rotating function. After the first rotation is complete with the lubrication of the nostrils and deep cleansing thereof, the cotton square's rotation is reversed with the compressing of the left button for the cleansing of excess particles within the nostrils.

A button placed in the center of the Wipe n′ Twirl, desists all rotation and immediately sets the tube and cotton square in start position. The invention not only provides deep cleaning for the nostrils with accurately fitting lubricated cotton squares, but due to the 100 percent natural hypoallergenic fluid this invention contains, it cures allergies and sinus infections. To complete the varied functions of the hypoallergenic fluid this invention contains, The Wipe n′ Twirl's flexible fluid filled tube is removable by opening the side latch located on the right side of the invention. The invention hypoallergenic fluid heals cuts, heals burns, heals wounds, heals cold sores, rebuilds the health of hair, rebuilds healthy skin cells, removes make-up, and heals acne.

The invention possesses hypoallergenic fluids that simultaneously and effectively, provides hygienic care for the nose and various parts of the body. Thus, the Wipe n′ Twirl strengthens the nasal filters; a body's first line of defense against disease.

### BRIEF DESCRIPTION OF THE DRAWINGS

For a fuller understanding of the nature and objects of the invention, reference should be made to the following detailed description taken in conjunction with the accompanying drawings, in which:

FIG. 1 is a pictorial view of the front invention.

FIG. 2 is a pictorial view of the back invention.

FIG. 3 is a pictorial view of the varied internal operating parts of the invention.

### DETAILED DESCRIPTION OF THE PREFERRED EMBODIMENT

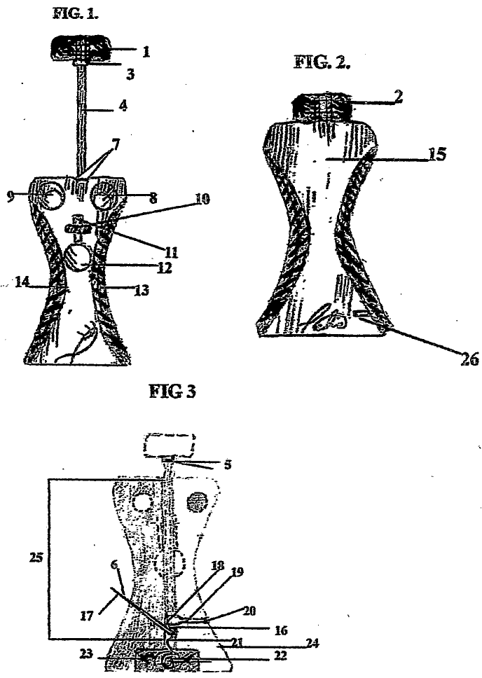

With reference to the drawings and, in particular, with reference to FIGS. 1-3, the WiPe n′ Twirl comprises a receptacle body indicated generally in FIG. 2 at 15, fabricated as an integral molding of an insulate material, preferably a titanium stainless steel material. the side hand grips made of rubber are depicted in FIG. 1 at 14, located on the front-left side of the products' body, and a front-right grip at 13.

The body is defined by a track rotatory at 7 allows object 4 to rotate precise area of rotation, the on button at 8 is located on the front-right side of the products' body, a reverse button at 9 is located on the front-left side of the products' body, a cotton popper at 10 releases the cotton cleansers from the tube holder, a cotton popper shaft at 11 is a vertical shaft that allows upward and downward movement of the cotton popper, a stop button at 12, activates the descent object 21 in FIG. 3, thus bringing objects in FIG. 1 at 1,3,4, FIG. 2 at 2, and FIG. 3 at 5,6,16, and 17, to the state of rest.

As depicted in FIG. 1 and FIG. 3, cleansing attachments includes a fluid filled rubber tube at 4, which allows fluid to flow freely into the entry point of object 5, and to continue through object into objects 1 and 2. The cotton cleanser at 1 is the front side of the dual cotton cleansers. The cotton cleansers at 1 receives the fluid from object 4, which begins the first cleansing activity. FIG. 2, depicts the reverse side of the cotton cleanser at 2, which is for excess cleansing and drying of the nostrils, and the insertion point of the travel power adapter at 26, which is for the stationary powering of the product without the use of the CR2025 lithium battery. The cotton connector at 3 is fused to 1, the tube connector at 5 and tube magnet at 6 are fused to 4 as seen in FIG. 1 and FIG. 3. When in operation 4 and 1 will extend from a position of rest.

As depicted in FIG. 3, the tube shaft at 25 secure holds the rubber fluid filled tube. The rubber fluid filled tube is placed into the tube holder at 16 and is magnetically secured onto tube holder cookie dial at 17. The tube holder cookie dial in FIG. 3 at 17, rotates objects in FIG. 1 at 1,3,4, and FIG. 2 at 2, and FIG. 3, at 5, and 6, upward-clockwise and counter clockwise. The tube is secured within in the constraints of the tube holder at 25.

As depicted in FIG. 3, fluid is released into the cotton by a tube constrictor head at 18, the tube constrictor body at 19 is securely fused into the tube constrictor holder at 20. The lift wire at 21 lifts objects FIG. 1 at 1,3,4, FIG. 2 at 2, and FIG. 3 at 5,6,16 and 17. The cookie dial at 22 rotates objects depicted in FIG. 1 at 1,3,4, FIG. 2 at 2, and FIG. 3 at 5,6,16,17 and 21, clockwise and counter-clockwise. The base plate located at the base of the product at 23 houses the CR2025 and secures the cookie dial.

As depicted in FIG. 3, a latch located on the front right-end of the product at 24, opens the product for the insertion of the detachable cleansing items depicted in FIG. 1, at 1,3,4, and FIG. 3 at 5 and 6.

The embodiment of the invention in which an exclusive property or privilege is claimed are defined as follows:

1. A hand-held electronic nose cleaning device having left, middle, and right activity buttons, said electronic nose cleaner comprising a receptacle body, said body being defined by left and right soft grips, a cotton popper, a tube-shaft; securing a fluid filled tube, tube magnet connecting cotton square, a tube constrictor mounted on the right wall of the said electronic nose cleaner a t-holder, said t-holder being defined by a rubber structure, a t-holder cookie dial, a lift wire extends from the cookie dial upward into the said t-holder, the said cookie dial is mounted on a base plate located at the base of said electronic nose cleaner.

2. The electronic nose cleaner recited in claim 1 wherein said left button is is associated with the reversal function of the rubber tube and cotton square having touch conscience impenetrable soft glass material.

3. The electronic nose cleaner recited in claim 1 wherein said middle button is associated with the desist and descend of the lift wire with the reversal function of the rubber tube and cotton square having touch conscience impenetrable soft glass material.

4. The electronic nose cleaner recited in claim 1 wherein said right button is associated with the active and ascend function of the lift wire having touch conscience impenetrable soft glass material.

5. The electronic nose cleaner recited in claim 1 wherein said body is fabricated from stainless titanium steel material.

6. The electronic nose cleaner recited in claim 1 wherein said left and right soft grips are fabricated with grafted rubber material.

7. The electronic nose cleaner recited in claim 6 wherein said left and right soft grips have a curvature for left and right palm and thumb grip.

8. The electronic nose cleaner recited in claim 1 wherein said cotton popper positional orientation is in the middle of said body.

9. The electronic nose cleaner recited in claim 8 wherein said cotton popper have a rubber retractable release.

10. The electronic nose cleaner recited in claim 9 wherein said cotton popper releases connecting cotton square from the connect point of the fluid filled Tube.

11. The electronic nose cleaner recited in claim 1 wherein said tube-shaft is located in the inner core and center of said electronic nose cleaner.

12. A hand-held electronic nose cleaning device having left, middle, and right activity buttons, said electronic nose cleaner comprising a receptacle body, said body being defined by left and right soft grips, a cotton popper, a tube-shaft; securing a fluid filled tube, tube magnet, and connecting cotton square, a tube constrictor mounted on the right wall of the said electronic nose cleaner, a t-holder, said t-holder being defined by a rubber structure, a t-holder cookie dial, a lift wire extends from the cookie dial upward into the said t-holder, the said cookie dial is mounted on a base plate located at the base of said electronic nose cleaner.

13. The electronic nose cleaner recited in claim 12 wherein said tube-shaft enables an accurate gliding ascension with appropriate security.

14. The electronic nose cleaner recited in claim 12 wherein said fluid filled tube consists of a formulated hypoallergenic cleansing fluid.

15. The electronic nose cleaner recited in claim 12 wherein said tube magnet is fused to the base of the fluid filled tube.

16. The electronic nose cleaner recited in claim 15 wherein said tube magnet connects to the magnet of said tube holder.

17. The electronic nose cleaner recited in claim 12 wherein said cotton square are formulated from gauze and hatched style cotton material.

18. The electronic nose cleaner recited in claim 12 wherein said tube constrictor inner core provides accurate fitting of the fluid filled tube for the constricting of the tube and the discretion of the fluid into said cotton square.

19. The electronic nose cleaner recited in claim 12 wherein said t-holder is fused to the said lift wire and secures said fluid filled tube.

20. The electronic nose cleaner recited in claim 12 wherein said t-holder cookie dial is magnetically connected to said tube magnet.

21. The electronic nose cleaner recited in claim 12 wherein said lift wire is fused to the tube holder, said lift wire lifts and rotates said fluid filled tube and connected cotton square.

22. The electronic nose cleaner recited in claim 12 wherein said cookie dial secures said lift wire by fusion, said cookie dial rotates lift wire mounted on the said base plate.
